(19) United States
(12) Patent Application Publication
(10) Pub. No.: US 2026/0283684 A1
(43) Pub. Date: Sep. 24, 2026

(54) METHODS AND APPARATUS FOR TREATMENT OF BODY TISSUE

(71) Applicant: RINDA SAMA, Las Vegas, NV (US)

(72) Inventor: RINDA SAMA, Las Vegas, NV (US)

(21) Appl. No.: 19/572,220

(22) Filed: Mar. 19, 2026

Related U.S. Application Data
(60) Provisional application No. 63/775,246, filed on Mar. 20, 2025.

(51) Int. Cl. A61B 18/14; A61B 17/00; A61B 17/32; A61B 18/00; G16H 30/20; G16H 50/20

(52) U.S. Cl. CPC A61B 18/1492; G16H 30/20; G16H 50/20; A61B 2017/00106; A61B 2017/00867; A61B 2017/320069; A61B 2018/00267; A61B 2018/00488; A61B 2018/00904; A61B 2018/1435

(57) ABSTRACT

A method for treating an undesirable human esophageal condition includes inserting an elongate treatment device including a distal portion into a nostril of an awake subject, the distal portion including an expandable energy delivery site having a low-profile configuration and an expanded configuration, advancing the distal portion of the device into the esophagus of the subject via the pharynx, expanding the expandable energy delivery site toward the expanded configuration at or in proximity to a target location at an inner wall of the esophagus of the subject, and delivering energy toward the target location with the expandable energy delivery site.

### INCORPORATION BY REFERENCE TO ANY PRIORITY APPLICATIONS

This application claims the benefit of priority to U.S. Provisional Patent Application No. 63/775,246, filed on Mar. 20, 2025, which is herein incorporated by reference in its entirety for all purposes. Priority is claimed pursuant to 35 U.S.C. § 119.

### BACKGROUND OF THE INVENTION

### FIELD OF THE INVENTION

The field of the invention generally relates to treatment of diseases of the esophagus and treatment probes and systems for providing the treatment. The field relates to nasal delivery of diagnostic and therapeutic devices for treatment of the esophagus.

### SUMMARY OF THE INVENTION

In one embodiment of the present disclosure, a method for treating an undesirable human esophageal condition includes inserting an elongate treatment device including a distal portion into a nostril of an awake subject, the distal portion including an expandable energy delivery site having a low-profile configuration and an expanded configuration, advancing the distal portion of the device into the esophagus of the subject via the pharynx, expanding the expandable energy delivery site toward the expanded configuration at or in proximity to a target location at an inner wall of the esophagus of the subject, and delivering energy toward the target location with the expandable energy delivery site.

In another embodiment of the present disclosure, a system for treating cancerous or potentially pre-cancerous disease in the esophagus of a patient includes a probe including an elongate shaft having a proximal end and a distal end, the shaft comprising a distal portion comprising one or more active elements, the distal portion having a substantially linear or curvilinear configuration configured for insertion into a nostril of a nose of a subject and an expanded configuration configured to contact or approximate a target location at an inner wall of the esophagus of the subject.

### BRIEF DESCRIPTION OF THE DRAWINGS

FIG. 1 is a partial sectional view of a treatment device being inserted into the nostril of a patient, according to an embodiment of the present disclosure.

FIG. 2 is a partial sectional view of the treatment device being inserted into a target area of the esophagus of the patient, according to an embodiment of the present disclosure.

FIG. 3 is a partial sectional view of the treatment device taking an expanded configuration within the target area of the esophagus of the patient, according to an embodiment of the present disclosure.

FIG. 4 is a perspective view of a treatment device, according to an embodiment of the present disclosure.

FIG. 5 is a perspective view of a treatment device having a distal helical shape, according to an embodiment of the present disclosure.

FIG. 6 is a detailed perspective view of the device of FIGS. 4-5.

FIG. 7 is a perspective view of a treatment device having a tapered helix, according to an embodiment of the present disclosure.

FIG. 8 is a detailed perspective view of the device of FIG. 7.

FIG. 9 is a detailed perspective view of the device of FIG. 7.

FIG. 10 is a perspective view of a treatment device having a substantially planar spiral, according to an embodiment of the present disclosure.

FIG. 11 is a detailed perspective view of the device of FIG. 10.

FIG. 12 is a detailed perspective view of the device of FIG. 10.

FIG. 13 is a detailed perspective view of a treatment device having a distal helical shape, according to an alternative embodiment of the present disclosure.

FIG. 14 is a detailed perspective view of a treatment device having a distal substantially planar spiral, according to an alternative embodiment of the present disclosure.

### DETAILED DESCRIPTION OF THE ILLUSTRATED EMBODIMENTS

Numerous maladies can affect the esophagus of patients, including, but not limited to, gastroesophageal reflux disease (GERD), achalasia, Barrett's esophagus, esophageal cancer, eosinophilic esophagitis, esophageal diverticulum, esophageal muscle spasm, esophageal stricture, and hiatal hernia. Multiple embodiments are disclosed herein for treating any of these maladies in patients via insertion in a naso-esopageal manner. By bypassing the stimulation of the posterior pharyngeal wall, tonsils, or base of the tongue, the gag reflex, or pharyngeal reflex, is avoided. Thus, sedation is not required, making any one of these treatments a quick and simple outpatient procedure. Furthermore, embodiments of apparatus and methods for disease detection, identification, characterization, planning, and treating are disclosed. Any combination of one or more of the features disclosed herein are incorporated into a single deliverable device, or into an efficient to use system, such that in some cases, all or the majority of the diagnostic and therapeutic steps can be performed in a single procedure.

In one embodiment, a method for treating a patient includes inserting an elongate treatment device including a distal portion into a nostril of an awake subject, the distal portion including an expandable energy delivery site having a low-profile configuration and an expanded configuration; advancing the distal portion of the device into the esophagus of the subject via the pharynx; expanding the expandable energy delivery site toward the expanded configuration at or in proximity to a target location at an inner wall of the esophagus of the subject; and delivering energy toward the target location with the expandable energy delivery site.

In some embodiments, the expandable energy delivery site includes an expandable basket. In some embodiments, the basket includes a shape-memory alloy. In some embodiments, the shape-memory alloy comprises a nickel-titanium alloy. In some embodiments, the shape-memory alloy in the low-profile configuration of the expandable energy delivery site is in a stress-induced Martensite phase. In some embodiments, the expandable energy delivery site includes an elongate shaft in the low-profile configuration. In some embodiments, the expandable energy delivery site includes a shape configured to approximate the shape of the inner wall of the esophagus in the expanded configuration. In some embodiments, the shape of the expandable delivery site in the expanded configuration is a spiral. In some embodiments, the shape of the expandable delivery site in the expanded configuration is a substantially planar spiral. In some embodiments, the shape of the expandable delivery site in the expanded configuration is a helix. In some embodiments, the helix has a substantially constant diameter. In some embodiments, the helix has a diameter that changes over its length. In some embodiments, a distal diameter of the helix is greater than a proximal diameter of the helix. In some embodiments, the diameter of the helix changes linearly along a longitudinal axis of the helix.

In some embodiments, the expandable energy delivery site in its low-profile configuration has greater flexibility than it does in its expanded configuration. In some embodiments, the elongate treatment device does not include a balloon. In some embodiments, the expandable energy delivery site includes one or more ultrasound transducers. In some embodiments, the one or more ultrasound transducers are each configured to vibrate a frequency of between about 20 kHz and about 50 kHz.

In some embodiments, the method includes treating cells at the target location with the one or more ultrasound transducers. In some embodiments, the method includes obtaining an ultrasound scan at the target location with the one or more ultrasound transducers. In some embodiments, the method includes, analyzing data and/or an image of the ultrasound scan with an artificial intelligence (AI) unit. In some embodiments, the method includes, determining one or more parameters of a treatment regimen provided by the expandable energy delivery site from the analysis of the data and/or the image by the artificial intelligence (AI) unit. In some embodiments, the one or more parameters include an energy delivery parameter. In some embodiments, the one or more parameters include a physical dimension of expandable energy delivery site in its expanded configuration. In some embodiments, the analysis of the data and/or the image outputs a probability of whether the inner wall of the esophagus of the subject is likely to include cancer. In some embodiments, the analysis includes a multi-color depiction of a portion of the esophagus. In some embodiments, the delivering energy toward the target location and obtaining an ultrasound scan at the target location are both performed in a single procedure. In some embodiments, the single procedure is a single outpatient procedure. In some embodiments, the one or more parameters include an energy delivery parameter.

In some embodiments, the expandable energy delivery site includes one or more electrodes. In some embodiments, the one or more electrodes are configured to provide pulses at 500 Volts to 1500 Volts. In some embodiments, the method includes treating cells at the target location with the one or more electrodes. In some embodiments, the method includes obtaining an impedance mapping at the target location with the one or more electrodes. In some embodiments, the method includes, analyzing data and/or an image of the impedance mapping with an artificial intelligence (AI) unit. In some embodiments, the method includes determining one or more parameters of a treatment regimen provided by the expandable energy delivery site from the analysis of the data and/or the image by the artificial intelligence (AI) unit. In some embodiments, the one or more parameters include an energy delivery parameter. In some embodiments, the one or more parameters include a physical dimension of expandable energy delivery site in its expanded configuration. In some embodiments, the analysis of the data and/or the image outputs a probability of whether the inner wall of the esophagus of the subject is likely to include cancer. In some embodiments, the analysis includes a multi-color depiction of a portion of the esophagus. In some embodiments, the delivering energy toward the target location and obtaining an impedance mapping at the target location are both performed in a single procedure. In some embodiments, the single procedure is a single outpatient procedure.

In one embodiment, a system for treating a patient includes an elongate shaft having a proximal end and a distal end, the shaft including a distal portion including one or more active elements, the distal portion having a substantially linear or curvilinear configuration configured for insertion into a nostril of a nose of a subject and an expanded configuration configured to contact or approximate a target location at an inner wall of the esophagus of the subject. In some embodiments, the one or more active elements include one or more ultrasound transducers. In some embodiments, the one or more active elements include one or more piezoelectric elements. In some embodiments, the one or more ultrasound transducers are configured to vibrate a frequency of between about 20 kHz and about 50 kHz. In some embodiments, the one or more active elements include one or more electrodes. In some embodiments, the one or more electrodes are configured to provide pulses at 500 Volts to 1500 Volts. In some embodiments, the one or more electrodes are configured to obtain an impedance mapping at the target location.

In some embodiments, the system includes a microprocessor configured to determine one or more parameters of a treatment regimen provided by the one or more active elements from the analysis of the data and/or the image. In some embodiments, the system further includes an artificial intelligence (AI) unit configured to determine one or more parameters of a treatment regimen provided by the one or more active elements from the analysis of the data and/or the image. In some embodiments, the one or more parameters include an energy delivery parameter. In some embodiments, the one or more parameters include a physical dimension of expandable energy delivery site in its expanded configuration. In some embodiments, the analysis of the data and/or the image outputs a probability of whether the inner wall of the esophagus of the subject is likely to include cancer. In some embodiments, the analysis includes a multi-color depiction of a portion of the esophagus. In some embodiments, the expanded configuration comprises a spiral shape. In some embodiments, the spiral is a substantially planar spiral. In some embodiments, the expanded configuration comprises a helical shape. In some embodiments, the helix has a substantially constant diameter. In some embodiments, the helix has a diameter that changes over its length. In some embodiments, a distal diameter of the helix is greater than a proximal diameter of the helix. In some embodiments, the diameter of the helix changes linearly along a longitudinal axis of the helix.

In some embodiments, the distal portion in its substantially linear or curvilinear configuration has greater flexibility than it does in its expanded configuration. In some embodiments, the probe does not include an inflatable balloon. In some embodiments, the distal portion includes an expandable basket. In some embodiments, the basket includes a shape-memory alloy. In some embodiments, the shape-memory alloy in the substantially linear or curvilinear configuration of the distal portion is in a stress-induced Martensite phase. In some embodiments, the shaft includes an internal lumen that extends at least through the distal portion. In some embodiments, the probe further includes an elongate member including a shape-memory alloy. In some embodiments, the shape-memory alloy is configured to be heated toward an Austenitic phase to form an expanded shape that forces the expanded configuration of the distal portion. In some embodiments, the probe further includes a mandrel configured to be moved longitudinally within the internal lumen. In some embodiments, a distal portion of the mandrel is configured to be advanced forward within the internal lumen at the distal portion of the shaft and to cause the substantially linear or curvilinear configuration. In some embodiments, the distal portion of the shaft is formed in a shape defining the expanded configuration, and wherein the distal portion of the mandrel is configured to be retracted within the internal lumen substantially out of the distal portion of the shaft, to allow the expanded configuration to be activated.

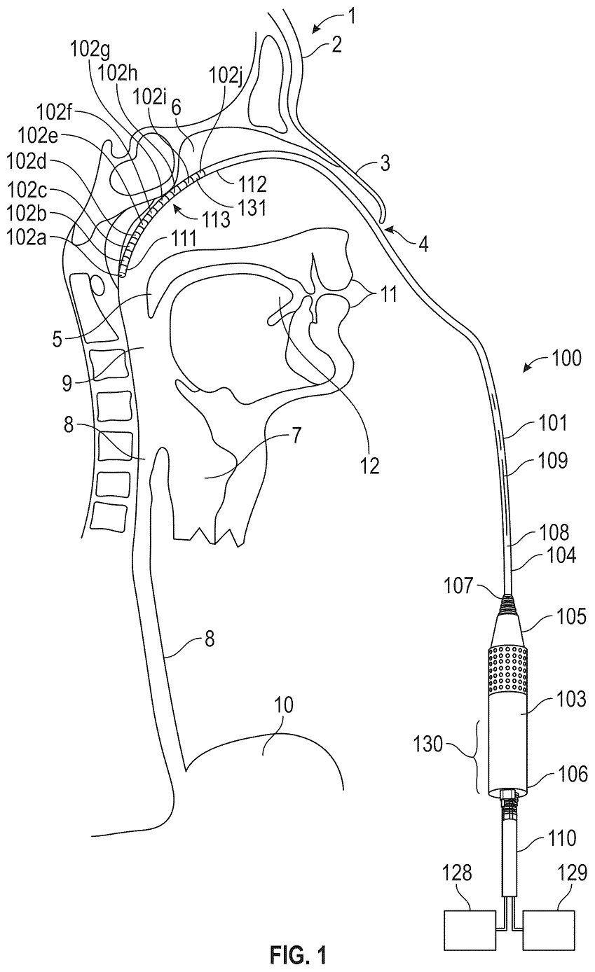

FIG. 1 illustrates the head 2 of a patient 1 including nose 3, lips 11, soft palate 5, and tongue 12. The esophagus 8 descends toward the stomach 10, and the trachea 7 descends toward the lungs (not shown). A probe 100 comprises a shaft 101 having a proximal end 104 and a distal end 111. A control handle 103, having a distal end 105 and a proximal end 106, is coupled to the proximal end 104 of the shaft 101 via a strain relief 107. The shaft 101 comprises a polymeric tubing having one or more lumens 108 configured to carry one or more elongate conductors 109. The conductors 109 can comprise electrically-insulated copper wires. One or more of the conductors 109 extend through the interior of the control handle 103, and proximally out via an extension cable 110, which can be coupled to a power unit 128 and/or a display unit 129. Alternatively, the control handle 103 can contain all of its own power, for example, via a battery 130. In some embodiments, the battery is rechargeable.

The distal end 111 of the shaft 101 and an intermediate location 112 of the shaft define and bracket a distal portion 113 of the shaft 101. The distal portion 113 includes one or more active elements 102. The embodiment of FIG. 1 is depicted has having ten active elements 102a-j, including a tip element 102a and linearly arrayed elements 102b-102j, which are arrayed proximally from the tip element 102a. There are nine spaces 131 on the shaft 101 at the distal portion 113, between each successive element 102. The active elements 102a-j can comprise ultrasound transducers (e.g., piezoelectric elements) configured for performing high-intensity ultrasound ablation and/or can comprise electrodes configured for performing pulse field ablation. As shown in FIG. 1, in a method of using a system including the probe 100, the distal portion 113 of the shaft 101 is inserted by a medical personnel into and through the nostril 4 of the nose 3 of the patient 1, along and in proximity to the ethmoid region 6 to reach a space 9 between the soft palate 5 and the esophagus 8.

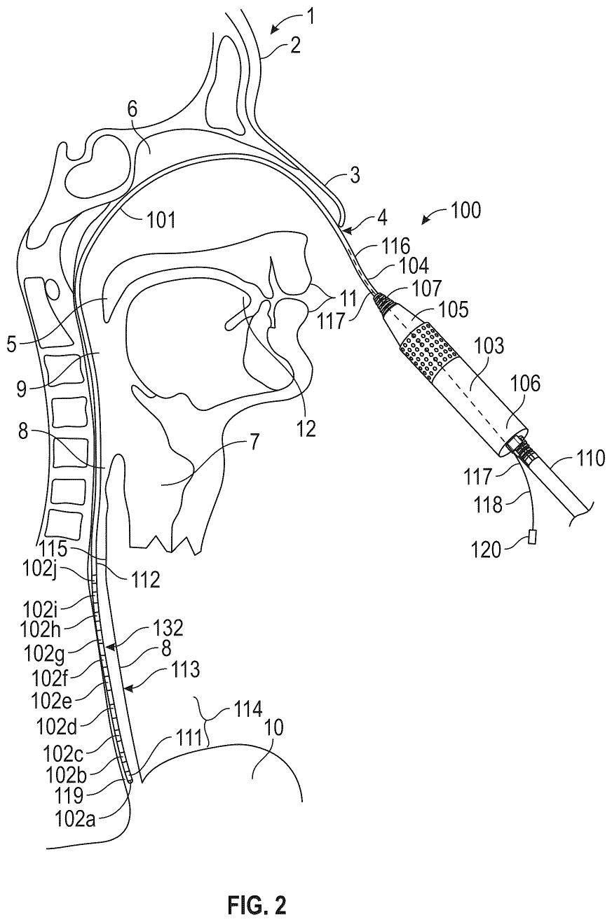

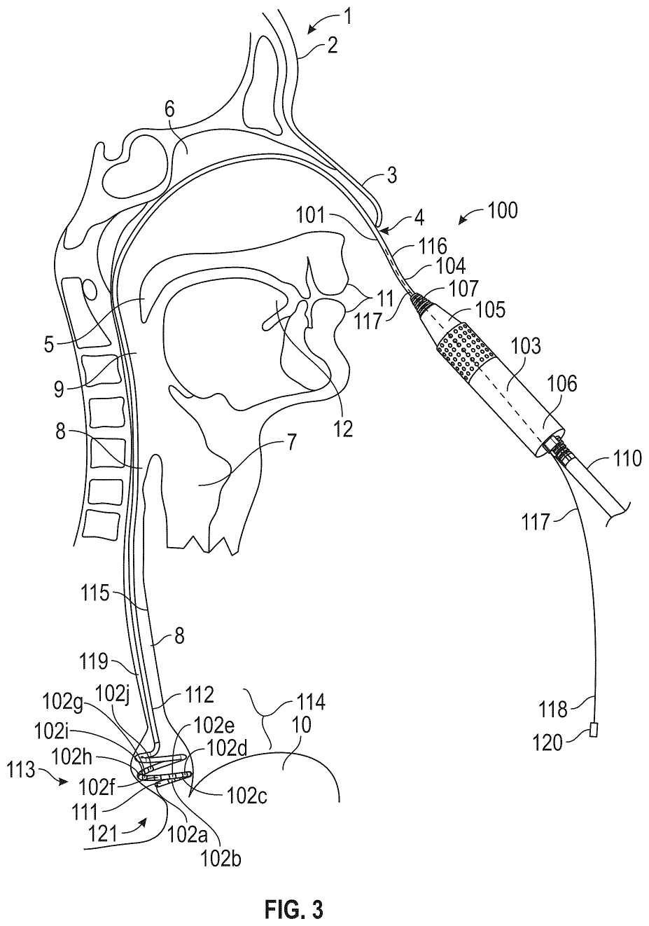

As shown in FIG. 2, the probe 100 is advanced by the user such that the distal portion moves past (inferiorly of) the space 9, and down the esophagus 8 to a target area 114 in the wall 115 of the esophagus 8. The shaft 101 further comprises a lumen 116 extending therethrough. The lumen 116 extends from the proximal end 104 to the distal end 111 and extends through the distal portion 113. A mandrel 117 is slideable within the lumen 116 and has a proximal end 118 and a distal end 119. The proximal end 118 is bonded to a handle 120 configured for grasping. In alternative embodiments, the proximal end can be coupled to an automated actuator for moving the mandrel distally and proximally within the lumen 116. In FIG. 2, the distal end 119 of the mandrel 117 is extended fully distally within the lumen 116, so that the stiffness of the mandrel 117 straightens the distal portion 113 of the shaft 101 from within the lumen 116. This is the substantially linear or curvilinear configuration 132 of the distal portion 113. The distal portion 113 of the shaft 101 is formed in this embodiment in a helical shape 121 (FIG. 3) by heat forming, or by a helical wire support within or on the shaft 101. FIG. 3 illustrates the expanded configuration of the distal portion 113. With the distal portion 113 in a generally chosen location adjacent to the target area 114 of the esophagus 8, a user grasps the handle 120 and pulls the mandrel 117 proximally so that the distal end 119 of the mandrel 117 is pulled fully proximally of the distal portion 113, within the lumen 116. This allows the distal portion 113 to change from the substantially linear or curvilinear configuration of FIG. 2, to the expanded configuration (e.g., helical shape 121) of FIG. 3. The user assures (e.g., via fluoroscopy) that the expanded distal portion 113, having the helical shape 121, is in the target area 114 and is sufficiently expanded to contact or closely approach the wall 115 of the esophagus 8 at the target area 114. The treatment of the esophagus 8 utilizing the linearly arrayed elements 102a-102j can now be performed. Diagnostics can also be performed utilizing the elements 102a-102j, when the embodiment allows for this, and when necessary. It can be seen in FIG. 3 how the elements 102a-102j are now located at the wall 115 of the esophagus 8 over a generally cylindrical surface area, to access a significant portion of the target area 114. As seen, this can cause temporary distension of the esophagus, in the same manner that food temporarily causes this distension when being passed therethrough.

After treatment and/or diagnosis is performed with the probe 100, the mandrel 117 can advanced forward within the lumen 116 by the user, to straighten out the distal portion 113, to make removal easier, though it can be possible to remove the entire probe 100 without any straightening. The probe 100 is then carefully removed from the esophagus and the nostril, by pulling on the shaft 101 directly or pulling the shaft 101 from the handle 103.

The expanded configuration of the distal portion 113 can be configured in many different manners, to optimized coverage of the target areas 114 of the esophagus 8. These target areas 114 can sometimes be long, sometimes short, sometimes focal, sometimes diffuse. In some cases, there may be certain areas of the esophagus 8 that need to be avoided for contact and/or treatment. Some patients may have an esophageal reconstruction area, and other patients may suffer from esophageal weakness, such as in Marfan's Syndrome.

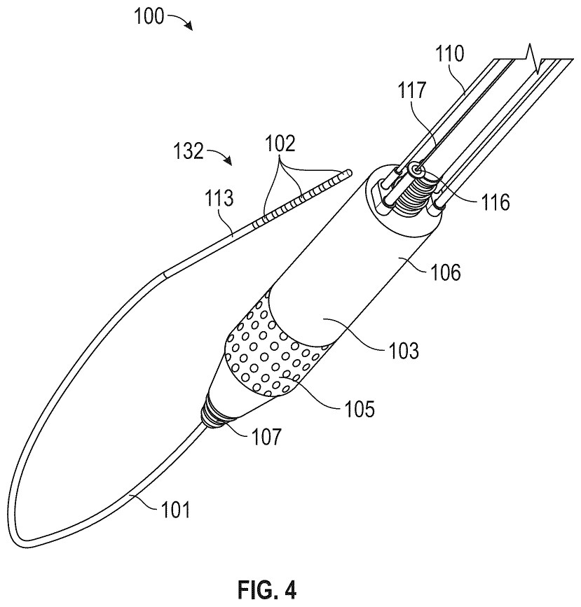

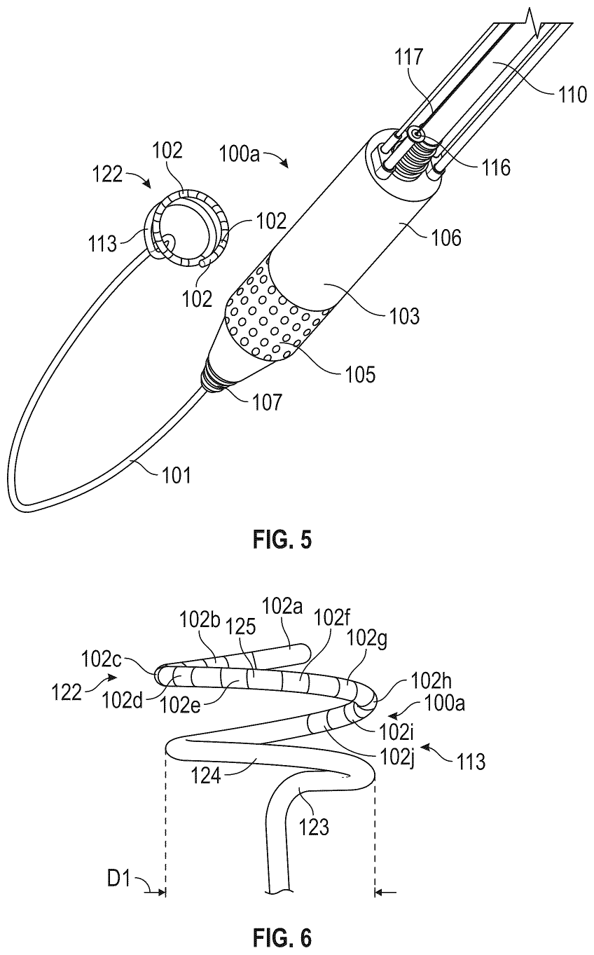

FIG. 4 shows a detail of a probe 100 with its distal portion 113 in the substantially linear or curvilinear configuration 132. Subsequent figures show a variety of embodiments of probes 100 with different expanded configurations of the distal portions 113. FIGS. 5-6 illustrate a probe 100a comprising a helix 122 in the expanded configuration of its distal portion 113. The helix 122 maintains substantially the same outer diameter D1 with very little taper, except, for example, at the lead-in proximal portion 123. The linearly arrayed elements 102a-102j, when the distal portion 113 takes the expanded configuration, become helically arrayed. This, when the term “linearly arrayed” is used herein, it refers to the configuration on the shaft 101 of the probe 100, when in a substantially straight shape. The helical presentation of the elements 102a-102j allows the elements 102a-102j to engage with the tissue of the wall 115 of the esophagus 8, when the distal portion 113 is in the expanded configuration. In some embodiments, the elements 102a-102j are distributed evenly around both full turns 124, 125 of the helix 122. However, in the embodiment of FIGS. 5-6, the elements 102a-102j are substantially located on the distal turn 125 and not the proximal turn 124. In this embodiment, the tip element 102a is longer than the other elements 102b-102j. However, in other embodiments, the lengths can be generally the same, or the other elements 102b-102j can be longer than the tip element 102a. In some embodiments, the outer diameter D1 is configured to be between about 10 mm and about 40 mm, or between about 20 mm and about 35 mm, or between about 15 mm and about 30 mm, or between about 20 mm and about 30 mm.

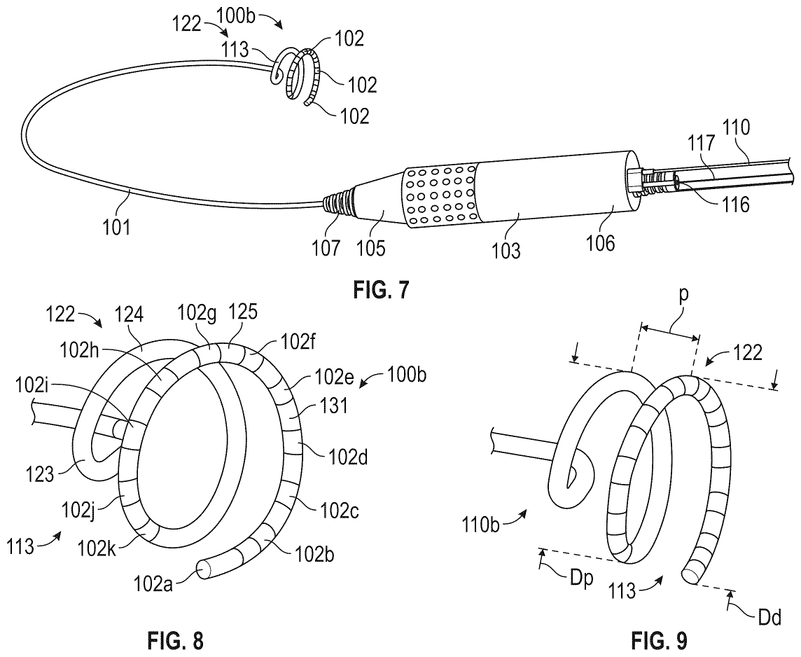

FIGS. 7-9 illustrate a probe 100b comprising a tapered helix 122 in the expanded configuration of its distal portion 113. The helix 122 diameter Dx tapers to a distally-located diameter Dd that is larger than a proximally-located diameter Dp. The linearly arrayed elements 102a-102j, when the distal portion 113 takes the expanded configuration, become spirally or helically arrayed. The taper can be useful if the esophagus of a patient tapers down from the stomach toward the head. This is particularly evident when the elements 102 continue well into the smaller diameter portions of the helix 122. Thus, there can be some matching between the tapered helix 122 and the esophagus 8 diameter. In other cases, where there is no substantial taper of the esophagus in the target area 114, the larger diameter Dd can in some cases serve as more of an anchor of the probe 100 to the esophagus 8. In some embodiments, the elements 102a-102k are distributed evenly around both full turns 124, 125 of the helix 122. However, in the embodiment of FIGS. 7-9, the elements 102a-102k are substantially located on the distal turn 125 and not the proximal turn 124. It should be noted that the probe 100a has ten elements 102a-102j, while the probe 100b has eleven elements 102a-102k. Other numbers or elements 102 can be chosen, from one to 20, or from one to 50, or even more. The pitch p between turns 124, 125 can be between about 1 mm and about 5 mm, or between 1 mm and 3 mm. The number of full turns 124, 125 can range from ½ to 30, or 2 to 10.

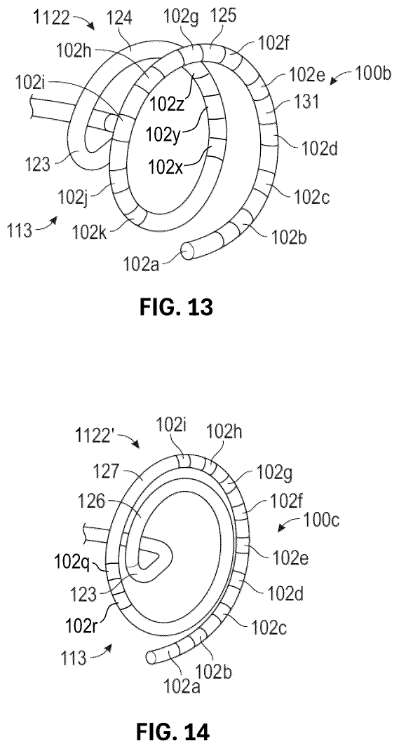

FIG. 13 illustrates an alternative helix 1122, that further includes elements 102x-z that are carried on the proximal turn 124. The additional elements 102x-z provide capability for treatment at a different longitudinal location of the esophagus from the elements 102a-k.

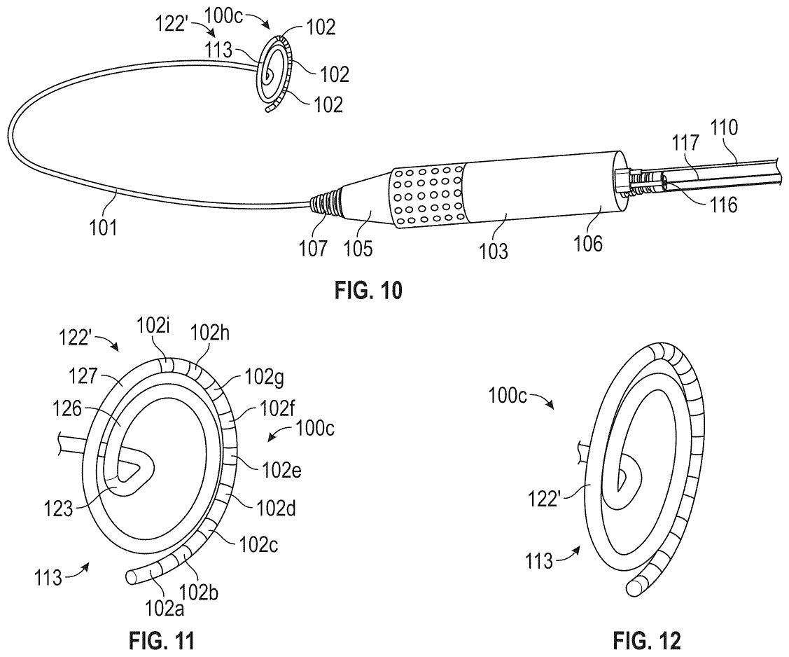

FIGS. 10-12 illustrate a probe 100c comprising a spiral 122′ in the expanded configuration of its distal portion 113. The spiral 122′ generally defines a plane. The planar shape is formed by a series of increasing turns 126, 127 or winds. Though continuous, the inner turn 126 and the outer turn 127 are indicated. In this embodiment, the probe 100c has nine elements 102a-102i. The elements 102a-102i are substantially on the outer turn 127. The inner turn 126 helps to support the outer turn 127, and thus force the elements 102a-102i into the wall 115 of the esophagus 8 at the target area 114. The planar shape of the spiral 122′ is substantially transverse to a longitudinal axis defined by the shaft 101 at a portion just proximal to the distal portion 113. In other embodiments, the spiral 122′ can be configured to be at a non-90° angle to the longitudinal axis of the shaft 101 just proximal to the distal portion 113. For example, the angle can be between about 40° and 140°, or between about 60° and 120°. The non-90° angulation can be appropriate for an esophagus 8 that is curved at or near the target area 114.

FIG. 14 illustrates an alternative spiral 1122′, that further includes elements 102q-r that are circumferentially spaced from the elements 102a-i. The additional elements 102q-r provide capability for treatment at a diametrically opposite side of the esophagus from the elements 102a-i.

The ranges disclosed herein also encompass any and all overlap, sub-ranges, and combinations thereof. Language such as “up to,” “at least,” “greater than,” “less than,” “between,” and the like includes the number recited. Numbers preceded by a term such as “approximately”, “about”, and “substantially” as used herein include the recited numbers (e.g., about 10%=10%), and also represent an amount close to the stated amount that still performs a desired function or achieves a desired result. For example, the terms “approximately”, “about”, and “substantially” may refer to an amount that is within less than 10% of, within less than 5% of, within less than 1% of, within less than 0.1% of, and within less than 0.01% of the stated amount.

For purposes of the present disclosure and appended claims, the conjunction “or” is to be construed inclusively (e.g., “an apple or an orange” would be interpreted as “an apple, or an orange, or both”; e.g., “an apple, an orange, or an avocado” would be interpreted as “an apple, or an orange, or an avocado, or any two, or all three”), unless: (i) it is explicitly stated otherwise, e.g., by use of “either . . . or,” “only one of,” or similar language; or (ii) two or more of the listed alternatives are mutually exclusive within the particular context, in which case “or” would encompass only those combinations involving non-mutually-exclusive alternatives. For purposes of the present disclosure and appended claims, the words “comprising,” “including,” “having,” and variants thereof, wherever they appear, shall be construed as open-ended terminology, with the same meaning as if the phrase “at least” were appended after each instance thereof.

1-74. (canceled)

75. A method for treating an undesirable human esophageal condition, comprising:
inserting an elongate treatment device comprising a distal portion into a nostril of an awake subject, the distal portion comprising an expandable energy delivery site having a low-profile configuration and an expanded configuration;
advancing the distal portion of the device into the esophagus of the subject via the pharynx;
expanding the expandable energy delivery site toward the expanded configuration at or in proximity to a target location at an inner wall of the esophagus of the subject; and
delivering energy toward the target location with the expandable energy delivery site.

76. The method of claim 75, wherein the awake subject is not under the effect of a sedative.

77. The method of claim 75, wherein the subject has been diagnosed with Barrett's esophagus.

78. The method of claim 75, wherein the subject has been diagnosed with esophageal cancer.

79. The method of claim 75, wherein the subject has been diagnosed with one or more of the afflictions in the list consisting of: gastrointestinal reflux disease (GERD), achalasia, eosinophilic esophagitis, esophageal diverticulum, esophageal muscle spasm, esophageal stricture, and hiatal hernia.

80. The method of claim 75, wherein the expandable energy delivery site comprises an expandable basket.

81. The method of claim 80, wherein the basket comprises a shape-memory alloy.

82. The method of claim 81, wherein the shape-memory alloy in the low-profile configuration of the energy delivery site is in a stress-induced Martensite phase.

83. The method of claim 75, wherein the expandable energy delivery site comprises an elongate shaft in the low-profile configuration.

84. The method of claim 83, wherein the expandable energy delivery site in the expanded configuration comprises a shape configured to engage the inner wall of the esophagus.

85. The method of claim 84, wherein the shape of the expandable delivery site in the expanded configuration comprises a helix.

86. The method of claim 85, wherein the helix has a substantially constant outer diameter.

87. The method of claim 85, wherein the helix has an outer diameter that changes over its length.

88. The method of claim 87, wherein a distal outer diameter of the helix is greater than a proximal diameter of the helix.

89. The method of claim 87, wherein the outer diameter of the helix changes linearly along a longitudinal axis of the helix.

90. The method of claim 83, wherein the expandable energy delivery site in the expanded configuration comprises a shape configured to approximate the shape of the inner wall of the esophagus.

91. The method of claim 75, wherein the elongate treatment device does not comprise a balloon.

92. The method of claim 75, wherein the expandable energy delivery site comprises one or more ultrasound transducers.

93. The method of claim 92, wherein the one or more ultrasound transducers are configured to vibrate at a frequency of between 20 kHz and 50 kHz.

94. The method of claim 92, further comprising:
treating cells at the target location with the one or more ultrasound transducers.

95. The method of claim 92, further comprising:
obtaining an ultrasound scan at the target location with the one or more ultrasound transducers.

96. The method of claim 95, further comprising:
analyzing data and/or an image of the ultrasound scan with an artificial intelligence (AI) unit.

97. The method of claim 96, further comprising:
determining one or more parameters of a treatment regimen provided by the expandable energy delivery site from the analysis of the data and/or the image by the artificial intelligence (AI) unit.

98. The method of claim 96, wherein the analysis of the data and/or the image outputs a probability of whether the inner wall of the esophagus of the subject is likely to include cancer.

99. The method of claim 75, wherein the expandable energy delivery site comprises one or more electrodes.

100. The method of claim 99, wherein the one or more electrodes are configured to provide pulses at 500 Volts to 1500 Volts.

101. The method of claim 99, further comprising:
obtaining an impedance mapping at the target location with the one or more electrodes.

102. The method of claim 101, further comprising:
analyzing data and/or an image of the impedance mapping with an artificial intelligence (AI) unit.

103. The method of claim 102, further comprising:
determining one or more parameters of a treatment regimen provided by the expandable energy delivery site from the analysis of the data and/or the image by the artificial intelligence (AI) unit.

104. The method of claim 102, wherein the analysis of the data and/or the image outputs a probability of whether the inner wall of the esophagus of the subject is likely to include cancer.
